(19) United States
(12) Patent Application Publication
(10) Pub. No.: US 2001/0047107 A1
(43) Pub. Date: Nov. 29, 2001

(54) Ester synthesis

(76) Inventors: Eric Nicholas Coker, Albuquerque, NM (US); Simon Frederick Thomas Froom, Snaith (GB); Warren John Smith, Feltham (GB)

Correspondence Address: NIXON & VANDERHYE P.C., 8th Floor, 1100 North Glebe Road, Arlington, VA, 22201-4714, US

(21) Appl. No.: 09/752,835

(22) Filed: Jan. 3, 2001

Related U.S. Application Data
(63) Continuation of application No. PCT/GB99/02099, filed on Jul. 1, 1999.

(30) Foreign Application Priority Data
Jul. 14, 1998 (GB) 9815117.8

(51) Int. Cl. C07C 67/04

(52) U.S. Cl. 560/247

(57) ABSTRACT

This invention relates to a process for the production of lower aliphatic esters, said process comprising reacting a lower olefin with saturated lower aliphatic mono-carboxylic acid in the vapour phase in the presence of a heteropolyacid catalyst, characterised in that the reactants are rendered substantially free of basic nitrogenous compounds prior to being brought into contact with the heteropolyacid catalyst.

The present invention relates to a process for the synthesis of esters by reacting an olefin with a lower carboxylic acid in the presence of an acidic catalyst.

It is well known that olefins can be reacted with lower aliphatic carboxylic acids to form the corresponding esters. One such method is described in GB-A-1259390 in which an ethylenically unsaturated compound is contacted with a liquid medium comprising a carboxylic acid and a free heteropolyacid of molybdenum or tungsten. This process is a homogeneous process in which the heteropolyacid catalyst is unsupported. A further process for producing esters is described in JP-A-05294894 in which a lower fatty acid is esterified with a lower olefin to form a lower fatty acid ester. In this document, the reaction is carried out in the gaseous phase in the presence of a catalyst consisting of at least one heteropolyacid salt of a metal e.g. Li, Cu, Mg or K, being supported on a carrier. The heteropolyacid used is phosphotungstic acid and the carrier described is silica.

One of the problems with this process is that impurities present in the reactants and any inert gases used in the reaction have a tendency to deactivate the acid catalyst. That the impurities in the feedstock may be a problem has not been recognised until recently due to the diverse sources of the olefinic feedstock used in this process.

We have now found that the presence of basic nitrogen compounds even in relatively small amounts, for example, at or above 0.5 ppm in the fresh olefin component of the feed streams can be detrimental to the activity and lifetime of the heteropolyacid catalyst.

Accordingly, the present invention provides a process for the production of lower aliphatic esters said process comprising reacting a lower olefin with a saturated lower aliphatic mono-carboxylic acid in the vapour phase in the presence of a heteropolyacid catalyst characterised in that the reactants are rendered substantially free of basic nitrogenous compounds prior to being brought into contact with the heteropolyacid catalyst.

By the expression “substantially free of basic nitrogenous compounds” is meant here and throughout the specification that the feedstream (comprising the olefin, acetic acid and any water or ether recycled to the feedstream including any nitrogeneous inert gases used during the reaction) to the reactor has less than 0.5 ppm, preferably less than 0.1 ppm of basic nitrogen compounds in the fresh or recycled olefin (e.g. ethylene) component of the feedstreams prior to the feedstream entering the reactor inlet. Specific examples of such a nitrogenous compounds are ammonia, alkyl amines and aryl amines including polyalkylene polyamine and polyarylene polyamines.

The basic nitrogen compounds present as impurities in particular are detrimental to the acid catalyst and can cause deactivation. These impurities are usually present in the olefin feed such as e.g. ethylene to the reaction. The amount of this nitrogenous impurity present would depend upon the source of the olefin used in the feedstream.

The basic nitrogenous compounds present as impurities are believed to cause deactivation of the heteropolyacid catalyst. Such impurities may be removed from the feedstreams by a number of techniques. One such technique uses, for example, a guard bed capable of absorbing/adsorbing such impurities from the feedstreams. The guard bed suitably comprises an acidic material such as e.g. alumina (γ-alumina, bentonite), molecular sieves (e.g. zeolites) or ion-exchange resins. These materials may be used in any suitable form, for example, as powders, pellets or extrudates. A preferred method of removing basic nitrogenous compounds from a lower olefin, which comprises a second aspect of the invention, comprises contacting said lower olefin with an acid-zeolite adsorbent material. The lower olefin may form part of a feedstream, which further comprises a saturated, lower aliphatic mono-carboxylic acid. Suitable zeolite materials include H-mordenite and H-Y.

In the reaction, the olefin reactant used is suitably ethylene, propylene or mixtures thereof Where a mixture of olefins is used, the resultant product will inevitably be a mixture of esters. The source of the olefin reactant used may be a refinery product or a chemical grade olefin which invariably contains some alkanes admixed therewith.

The saturated, lower aliphatic mono-carboxylic acid reactant is suitably a C1-C4 carboxylic acid and is preferably acetic acid.

The reaction may be carried out in a plurality of reactors set up in series such that the reactant gases exiting from a first reactor are fed as the feed gas to a second reactor and so on for subsequent reactors, and an aliquot of the reactant monocarboxylic acid is introduced into the feed gas to the second and subsequent reactors so as to maintain the olefin to monocarboxylic acid ratio in the feed gas to each of the second and subsequent reactors within a pre-determined range.

Thus, the mole ratio of olefin to the lower monocarboxylic acid in the reactant gases fed to the first reactor is suitably in the range from 1:1 to 18:1, preferably from 10:1 to 14:1. During the reaction, when the reactant gases come into contact with the heteropolyacid in a catalyst bed, at least some of the acid is used up to form the ester in an exothermic reaction and the mole ratio of olefin to monocarboxylic acid increases considerably from a starting ratio of 12:1 to about 30:1 in the exit gases from the final reactor. Where the reaction is carried out in a plurality of reactors set up in series, the exit gases from the first reactor are fed as the feed gas to the second reactor and the exit gases from the second reactor are fed as the feed gas to the third reactor and so on. When using such a series of reactors, the olefin to monocarboxylic acid mole ratio in the feed gas to the second and subsequent reactors is seriously depleted due to the acid being used up in the formation of the ester. This mole ratio of olefin to monocarboxylic acid is brought to the desired range by injecting further aliquots of the monocarboxylic acid to the feed gas prior to its entry into each of the second and subsequent reactors. In the case of the manufacture of ethyl acetate from ethylene and acetic acid, this range of mole ratios of ethylene to acetic acid in the reactant gases fed to the first reactor is suitably in the range from 1:1 to 18:1, preferably from 10:1 to 14:1 and that of the feed gas to the second and subsequent reactors is suitably from 10:1 to 16:1. The addition of further aliquots of the monocarboxylic acid to the feed gas to the second and subsequent reactors should be sufficient to bring the mole ratio of the olefin to acid within this range of 10:1 to 16:1.

The plurality of reactors set up in series referred to above need not be a descrete set of individual reactors. The process of the present invention should work equally effectively if the reaction is carried out in one long reactor which has a plurality of catalyst beds set up in series and the acid is injected into the exit gases from the first bed to maintain the range of olefin to monocarboxylic acid within the predetermined range in the second and subsequent stages. In a typical reaction it is desirable to use about four reactors set up in series although this can be reduced or increased without adversely affecting the beneficial effect of the injection of the monocarboxylic acid to the feed gas to the second and subsequent catalyst beds or reactors.

The reactors used in this context are suitably run under adiabatic conditions. Due to the exothermic nature of the reaction, it may be necessary to cool the feed gases to the second and subsequent reactors so as to maintain the reaction temperature within the desired range. This cooling may be achieved either by inserting an intermediate cooling step between the each of the reactors and can be wholly or partially replaced by the injection of the acid into the feed gas to the second and subsequent reactors. The intermediate cooling step can also be used where a single long reactor which has a plurality of catalyst beds set up in series is used. In this latter case, the intermediate cooling step is used to cool the reactant gases entering the second and subsequent catalyst beds. Where a cooling step is used, this may be achieved e.g. by using one or more of heat exchanger tubes and by injection of the additional monocarboxylic acid reactant into the feed gases as described above.

The process of the present invention can be improved further by the addition of water as a component of the reaction mixture. The water added to the reaction mixture is suitably present in the form of steam and is capable of generating a mixture of esters and alcohols in the process. It has been found that the presence of water in the reaction mixture in an amount of 1-10 mole %, preferably from 3 to 7 mole %, e.g. 5 to 6.5 mole %, based on the total moles of acetic acid, olefin and water, enhances the stability of the catalyst and thereby enhances the efficiency of the process. Furthermore, the presence of water also reduces the selectivity of the process to undesired by-products such as e.g. oligomers and other unknowns, excluding diethyl ether and ethanol. Water addition may also be used to supplement the cooling of the feed gases to the second and subsequent reactors.

It has further been found that dosing the reaction mixture with amounts of a di-ether such as e.g. diethyl ether, as a co-feed also reduces the formation of undesirable by-products. The amount of di-ether co-fed is suitably in the range from 0.1 to 6 mole %, preferably in the range from 0.1 to 3 mole % based on the total reaction mixture comprising the olefin, the aliphatic carboxylic acid, water and diethyl ether. The di-ether co-fed may correspond to the by product di-ether from the reaction generated from the reactant olefin. Where a mixture of olefins is used, e.g. a mixture of ethylene and propylene, the di-ether may in turn be an unsymmetrical di-ether. The di-ether co-feed may thus be the by-product of the reaction which by-product is recycled to the reaction mixture.

The term “heteropolyacid” as used herein and throughout the specification in the context of the catalyst is meant to include the free acids. The heteropolyacids used to prepare the esterification catalysts of the present invention therefore include inter alia the free acids and co-ordination type partial acid salts thereof in which the anion is a complex, high molecular weight entity. Typically, the anion comprises 2-18 oxygen-linked polyvalent metal atoms, which are called peripheral atoms. These peripheral atoms surround one or more central atoms in a symmetrical manner. The peripheral atoms are usually one or more of molybdenum, tungsten, vanadium, niobium, tantalum and other metals. The central atoms are usually silicon or phosphorus but can comprise any one of a large variety of atoms from Groups I-VIII in the Periodic Table of elements. These include, for instance, cupric ions; divalent beryllium, zinc, cobalt or nickel ions; trivalent boron, aluminium, gallium, iron, cerium, arsenic, antimony, phosphorus, bismuth, chromium or rhodium ions; tetravalent silicon, germanium, tin, titanium, zirconium, vanadium, sulphur, tellurium, manganese nickel, platinum, thorium, hafnium, cerium ions and other rare earth ions; pentavalent phosphorus, arsenic, vanadium, antimony ions; hexavalent tellurium ions; and heptavalent iodine ions. Such heteropolyacids are also known as “polyoxoanions”, “polyoxometallates” or “metal oxide clusters”. The structures of some of the well known anions are named after the original researchers in this field and are known e.g. as Keggin, Wells-Dawson and Anderson-Evans-Perloff structures.

Heteropolyacids usually have a high molecular weight e.g. in the range from 700-8500 and include dimeric complexes. They have a relatively high solubility in polar solvents such as water or other oxygenated solvents, especially if they are free acids and in the case of several salts, and their solubility can be controlled by choosing the appropriate counter-ions. Specific examples of heteropolyacids that may be used as the catalysts in the present invention include:

12-tungstophosphoric acid—H₃[PW₁₂O₄₀].xH₂O

12-molybdophosphoric acid—H₃[PMo₁₂O₄₀].xH₂O

12-tungstosilicic acid—H₄[SiW₁₂O₄₀].xH2O

12-molybdosilicic acid—H₄[SiMo₁₂O₄₀].XH₂O

Cesium hydrogen tungstosilicate—Cs₃H[SiW₁₂O₄₀].xH₂O

The heteropolyacid catalyst whether used as a free acid or as a partial acid salt thereof is suitably supported, preferably on a siliceous support. The siliceous support is suitably in the form of extrudates or pellets.

The siliceous support used can be derived from an amorphous, non-porous synthetic silica especially fumed silica, such as those produced by flame hydrolysis of SiCl₄. Specific examples of such siliceous supports include Support 350 made by pelletisation of AEROSIL® 200 (both ex Degussa). This pelletisation procedure is suitably carried out by the process described in U.S. Pat. No. 5,086,031 (see especially the Examples) and is incorporated herein by reference. Such a process of pelletisation or extrusion does not involve any steam treatment steps and the porosity of the support is derived from the interstices formed during the pelletisation or extrusion step of the non-porous silica The silica support is suitably in the form of granules, beads, agglomerates, globules, extrudates or pellets having an average particle diameter of 2 to 10 mm, preferably 4 to 6 mm. The siliceous support suitably has a pore volume in the range from 0.3-1.2 ml/g, preferably from 0.6-1.0 m/g. The support suitably has a crush strength of at least 2 Kg force, suitably at least 5 Kg force, preferably at least 6 Kg and more preferably at least 7 Kg. The crush strengths quoted are based on average of that determined for each set of 50 beads/globules on a CHATTILLON tester which measures the minimum force necessary to crush a particle between parallel plates. The bulk density of the support is suitably at least 380 g/l, preferably at least 440 g/l.

The support suitably has an average pore radius (prior to use) of 10 to 500 Å preferably an average pore radius of 30 to 100 Å.

In order to achieve optimum performance, the siliceous support is suitably free of extraneous metals or elements which might adversely affect the catalytic activity of the system. The siliceous support suitably has at least 99% w/w purity, i.e. the impurities are less than 1% w/w, preferably less than 0.60% w/w and more preferably less than 0.30% w/w.

Other silica supports are the Grace 57 and 1371 grades of silica. In particular, Grace 57 grade silica has a bulk density of about 0.4 g/ml and a surface area in the range of 250-350 m²/g. Grace silica grade No. 1371 has an average bulk density of about 0.39 g/ml, a surface area of about 500-550 m²/g, an average pore volume of about 1.15 ml/g and an average particle size ranging from about 0.1-3.5 mm. These supports can be used as such or after crushing to an average particle size in the range from 0.5-2 mm and sieving before being used as the support for the heteropolyacid catalyst.

The impregnated support is suitably prepared by dissolving the heteropolyacid, which is preferably a tungstosilicic acid, in e.g. distilled water, and then adding the support to the aqueous solution so formed. The support is suitably left to soak in the acid solution for a duration of several hours, with periodic manual stirring, after which time it is suitably filtered using a Buchner funnel in order to remove any excess acid.

The wet catalyst thus formed is then suitably placed in an oven at elevated temperature for several hours to dry, after which time it is allowed to cool to ambient temperature in a desiccator. The weight of the catalyst on drying, the weight of the support used and the weight of the acid on support was obtained by deducting the latter from the former from which the catalyst loading in g/liter was determined.

Alternatively, the support may be impregnated with the catalyst using the incipient wetness technique with simultaneous drying on a rotary evaporator.

This supported catalyst (measured by weight) can then be used in the process of the invention. The amount of heteropolyacid deposited/impregnated on the support for use in the reaction is suitably in the range from 10 to 60% by weight, preferably from 20 to 50% by weight based on the total weight of the heteropolyacid and the support.

The reaction is carried out in the vapour phase suitably above the dew point of the reactor contents comprising the reactant acid, any alcohol formed in situ, the product ester and water as stated above. Dew point is the temperature at which condensation of a vapour of a given sample in air takes place. The dew point of any vaporous sample will depend upon its composition. The supported heteropolyacid catalyst is suitably used as a fixed bed in each reactor which may be in the form of a packed column. The vapours of the reactant olefins and acids are passed over the catalyst suitably at a GHSV in the range from 100 to 5000 per hour, preferably from 300 to 2000 per hour.

The reaction is suitably carried out at a temperature in the range from 150-200° C. within which range the entry temperature of the reactant gases is suitably from 160-180° C. and the temperature of the exit gases from each reactor is suitably 170-200° C. The reaction pressure is suitably at least 400 KPa, preferably from 500-3000 Kpa, more preferably about 1000 Kpa depending upon the relative mole ratios of olefin to acid reactant and the amount of water used.

The products of the reaction are recovered by e.g. fractional distillation. The esters produced, whether singly or as mixture of esters, may be hydrolysed to the corresponding alcohols or mixture of alcohols in relatively high yields and purity.

The process of the present invention is particularly suited to making ethyl acetate from ethylene and acetic acid by an addition reaction with optional recycle of any ethanol or diethyl ether formed.

### EXAMPLE

Adsorbent Bed for Removal of Basic Nitrogen Compounds from Gas Stream

Adsorbent Preparation

Adsorbents in the form of powders were pelletised, crushed and sieved to the size range 0.5-0.85 mm.

Adsorbents supplied as pellets or extrudates were crushed and sieved to 0.5-0.85 mm.

A range of acidic adsorbent materials suitable for the removal of basic nitrogen compounds from a gas stream were evaluated.

Adsorbent Evaluation

Between 2.5 and 20 ml of adsorbent particles (0.5-0.85 mm) were loaded into a tube (stainless steel, i.d. 20 mm). The adsorbent was activated by passing dry nitrogen through the tube (200 ml min⁻¹, 155° C., 0 barg) for 8-24 hours.

After activation, the tube was cooled to 25° C. and kept at that temperature for the duration of the adsorption experiment.

Ethylene containing ammonia (60 ppm) was passed through the adsorbent tube at a GHSV of 1,500-13,000 liters gas (liter adsorbent)⁻¹ h⁻¹ and at a pressure of 10-12 barg. Analysis of ammonia down-stream of the tube allowed determination of the capacity of the adsorbent for ammonia.

Results

The table summarises the process variables as well as the capacities and efficiencies of the various adsorbents tested.

Bentonite clay K306 was supplied by Süd Chemie, γ-alumina E3992 by Engelhard, zeolite H-mordenite by Laporte, zeolite SD-940 (H-Y) by Crosfield and zeolite CBV600 X16 (H-Y) by Zeolyst.

| | | Bed | | | | Capacity |
|---|---|---|---|---|---|---|
| | Type/ | size | NH₃ | Pressure | GHSV | [mmol |
| Adsorbent | form | [ml] | [ppm] | [barg] | [h⁻¹] | g⁻¹] |
| K306 | bentonite | 2.4 | 60 | 10 | 3125 | 0.26 |
| | pellets | | | | | |
| E3992 | γ-alumina | 5 | 60 | 10 | 1560 | 0.26 |
| | extrudates | | | | | |
| H- | zeolite | 2.8 | 60 | 10 | 6860 | 1.5 |
| mordenite | powder | | | | | |
| SD-940 | zeolite | 2.6 | 60 | 10 | 6920 | 2.6 |
| (H-Y) | powder | | | | | |
| CBV600 | zeolite | 2.6 | 60 | 12 | 12,900 | 1.7 |
| X16 (H-Y) | extrudates | | | | | |

The alumina and bentonite clay adsorbents possess capacities for ammonia of 0.26 mmol g⁻¹, while the acid-zeolite adsorbents possess higher capacities of between 1.2 and 2.6 mmol g⁻¹. In all of these experiments, efficiencies of ammonia adsorption of >99% were determined; thus ammonia levels were reduced from 60 ppm (upstream of the adsorbent) to 0.5 ppm or less (down-stream of the adsorbent).

1. A process for the production of lower aliphatic esters, said process comprising reacting a lower olefin with a saturated lower aliphatic mono-carboxylic acid in the vapour phase in the presence of a heteropolyacid catalyst, characterised in that the reactants are rendered substantially free of basic nitrogenous compounds prior to being brought into contact with the heteropolyacid catalyst.

2. A process as claimed in claim 1, wherein, prior to being brought into contact with the heteropolyacid catalyst, the concentration of nitrogenous compounds in the olefin reactant is less than 0.5 ppm.

3. A process as claimed in claim 1, wherein, prior to being brought into contact with the heteropolyacid catalyst, the concentration of nitrogenous compounds in the olefin reactant is less than 0.1 ppm.

4. A process as claimed in claim 1 wherein the nitrogenous compounds include ammonia, alkyl amines and aryl amines including polyalkylene polyamine and polyarylene polyamines.

5. A process as claimed in claim 1, wherein the nitrogenous compounds are removed by the use of a guard bed.

6. A process as claimed in claim 1, wherein the nitrogenous compounds are removed by use of a guard bed which comprises an acidic material consisting of alumina, a molecular sieve and/or ion-exchange resins.

7. A process as claimed in claim 6, wherein the acidic material is a molecular sieve in the form of a zeolite.

8. A process as claimed in claim 1 wherein the olefin reactant is ethylene, propylene or a mixture thereof.

9. A process as claimed in claim 1 wherein the saturated lower aliphatic mono-carboxylic acid reactant is a C₁-C₄ carboxylic acid.

10. A method of removing basic nitrogenous compounds from a lower olefin, said method comprising contacting said olefin with an acid-zeolite adsorbent material.

11. A method as claimed in claim 10, wherein the olefin forms part of a feedstream, which further comprises a saturated lower aliphatic mono-carboxylic acid.

12. A method as claimed in claim 10, wherein the concentration of nitrogenous compounds in the lower olefin is reduced to less than 0.5 ppm.

13. A method as claimed in claim 10, wherein the concentration of nitrogenous compounds in the lower olefin is reduced to less than 0.1 ppm.
